(19) United States
(12) Patent Application Publication
(10) Pub. No.: US 2026/0207908 A1
(43) Pub. Date: Jul. 23, 2026

(54) SEMAGLUTIDE-CONTAINING MICRONEEDLES AND MANUFACTURING METHOD THEREFOR

(71) Applicant: RAPHAS CO., LTD., Seoul (KR)

(72) Inventors: Do Hyeon JEONG, Seoul (KR); Jin Ah KIM, Seoul (KR); Ju Young JIN, Gimpo-si (KR)

(21) Appl. No.: 19/140,570

(22) PCT Filed: Oct. 4, 2023
(86) PCT No.: PCT/KR2023/015239

§ 371 Date: Jun. 18, 2025

(30) Foreign Application Priority Data
Dec. 23, 2022 (KR) 10-2022-0183687

(51) Int. Cl. A61M 37/00; A61K 38/26; A61K 47/38

(52) U.S. Cl. CPC A61M 37/0015; A61K 38/26; A61K 47/38; A61M 2037/0053

(57) ABSTRACT

The present disclosure relates to a composition for preparing a microneedle containing semaglutide and a biodegradable polymer material, a microneedle, and a method of preparing a microneedle. The composition for preparing a microneedle of the present disclosure can be used to prepare a microneedle maintaining a strength suitable for skin penetration while having a high loading amount of semaglutide. In addition, the microneedle of the present disclosure or a microneedle prepared by the preparation method of the present disclosure may deliver a sufficient amount of drug into the body even in a small amount, and when these microneedles include a drug-loaded layer and a base structural layer, the residual amount of semaglutide in the needles after application to the skin is small, thereby reducing the loss of semaglutide.

### TECHNICAL FIELD

The present disclosure relates to a microneedle containing semaglutide and a preparation method thereof.

### BACKGROUND ART

Semaglutide, as glucagon-like peptide 1 (GLP-1) receptor agonist, is used as a therapeutic agent for type 2 diabetes and obesity, and is classified as a long-term agonist.

Semaglutide is mainly used as an oral medication or an injectable medication. Semaglutide may be enzymatically degraded in the gastrointestinal tract and intestinal mucosa, and may be poorly absorbed by the intestinal mucosa. In addition, due to various barriers, such as first-pass metabolism in the liver, parenteral administration is preferable to oral administration. Meanwhile, in the case of injections, there are issues such as causing pain to patients who receive injections, risk of secondary infection, and generation of medical waste. In addition, self-injectable medications must be administered by patients themselves, which can cause fear and refusal, leading to poor medication compliance and subsequent reduction of therapeutic effects.

Accordingly, there is a need to develop a microneedle patch formulation of semaglutide, and in particular, there is a need for the development of a patch formulation that can reduce the loss of semaglutide by reducing the residual amount of semaglutide in the needle after application to the skin.

### DISCLOSURE OF EMBODIMENTS

### Technical Problem

The present disclosure aims to provide a composition for preparing a microneedle that maintains a strength suitable for skin penetration while having a high loading amount of semaglutide.

The present disclosure aims to provide a microneedle containing semaglutide, which has improved ease of administration compared to self-injectable therapeutic agents.

In addition, the present invention aims to provide a microneedle and a preparation method thereof, the microneedle being able to reduce the loss of semaglutide due to a small residual amount of semaglutide in the needle after application to the skin.

### Solution to Problem

1. A composition for preparing a microneedle, including semaglutide and a biodegradable polymer material.
2. The composition for preparing a microneedle of No. 1., wherein the biodegradable polymer material is at least one selected from the group consisting of hyaluronic acid, polyvinylpyrrolidone, chondroitin, hydroxyethyl cellulose, and hydroxypropyl methylcellulose.
3. The composition for preparing a microneedle of No. 1., wherein a weight ratio of semaglutide and the biodegradable polymer material is 1:1.1 to 2.9.
4. The composition for preparing a microneedle of No. 1., further including at least one selected from the group consisting of disodium phosphate (Na₂HPO₄) and sodium hydroxide (NaOH).
5. The composition for preparing a microneedle of No. 1., further comprising at least one selected from the group consisting of trehalose and dextran.
6. A microneedle including a drug-loaded layer including semaglutide and a biodegradable polymer material.
7. The microneedle of No. 6., wherein the biodegradable polymer material of the drug-loaded layer is at least one selected from the group consisting of hyaluronic acid, polyvinylpyrrolidone, chondroitin, hydroxyethyl cellulose, and hydroxypropyl methylcellulose.
8. The microneedle of No. 6., wherein a weight ratio of semaglutide and the biodegradable polymer material in the drug-loaded layer is 1:1.1 to 2.9.
9. The microneedle of No. 6., further including a base structural layer disposed on a lower part of the drug-loaded layer and including a biodegradable polymer material.
10. The microneedle of No. 9., wherein the base structural layer is formed using a viscous composition including a biodegradable polymer material and water.
11. The microneedle of No. 10., wherein the biodegradable polymer material of the viscous composition is included in an amount of 8 to 15 wt % based on the total weight of the viscous composition.
12. The microneedle of No. 9., wherein the biodegradable polymer material of the base structural layer is at least one selected from the group consisting of hyaluronic acid and carboxymethyl cellulose.
13. A method of preparing a microneedle, including: forming a drug-loaded layer by spotting a first viscous composition including semaglutide and a biodegradable polymer material on a first support.
14. The method of preparing a microneedle of No. 13., wherein the forming of the drug-loaded layer includes: spotting the first viscous composition at multiple points spaced apart from each other on the first support; tensioning spotted first viscous composition; and drying tensioned first viscous composition.
15. The method of preparing a microneedle of No. 14., wherein the tensioning of the first viscous composition is performed by contacting a second support with spotted first viscous composition and subsequently spacing the first support and the second support apart.
16. The method of preparing a microneedle of No. 14., wherein the forming of the drug-loaded layer further includes cutting dried first viscous composition.
17. The method of preparing a microneedle of No. 13., wherein the biodegradable polymer material of the first viscous composition is at least one selected from the group consisting of hyaluronic acid, polyvinylpyrrolidone, chondroitin, hydroxyethyl cellulose, and hydroxypropyl methylcellulose.
18. The method of preparing a microneedle of No. 13., wherein a weight ratio of semaglutide and the biodegradable polymer material in the first viscous composition is 1:1.1 to 2.9.
19. The method of preparing a microneedle of No. 13., further including forming a base structural layer by spotting a second viscous composition including a biodegradable polymer material on the first support, before the spotting of the first viscous composition on the first support, wherein the first viscous composition is spotted on the base structural layer.
20. The method of preparing a microneedle of No. 19., wherein the forming of the base structural layer includes: spotting the second viscous composition at multiple points spaced apart from each other on the first support; and drying spotted second viscous composition.
21. The method of preparing a microneedle of No. 19., wherein the second viscous composition includes a biodegradable polymer material and water.
22. The method of preparing a microneedle of No. 21., wherein the biodegradable polymer material of the second viscous composition is included in an amount of 8 to 15 wt % based on the total weight of the second viscous composition.
23. The method of preparing a microneedle of No. 19., wherein the biodegradable polymer material of the second viscous composition is at least one selected from the group consisting of hyaluronic acid and carboxymethyl cellulose.

### Advantageous Effects of Disclosure

The composition for preparing a microneedle of the present disclosure can be used to prepare a microneedle maintaining a strength suitable for skin penetration while having a high loading amount of semaglutide.

The microneedle of the present disclosure can reduce the risk of bleeding, risk of infection, pain, fear, etc. compared to a self-injectable therapeutic agent containing semaglutide, thereby increasing convenience of administration for patients and reducing medical waste.

In addition, the microneedle of the present disclosure has a sufficient strength to be inserted into the skin, and includes a base structural layer at the lower portion, such that the residual amount of semaglutide in the drug-loaded layer on the base structural layer after application to the skin is small, thereby reducing the loss of semaglutide.

### BRIEF DESCRIPTION OF DRAWINGS

FIG. 1 illustrates a structure of a microneedle according to exemplary embodiments.

FIGS. 2 and 3 show process flow diagrams illustrating a method of preparing a microneedle according to exemplary embodiments.

FIG. 4 is for explaining maximum width and height of a base structural layer of a microneedle according to exemplary embodiments.

FIG. 5 shows a pharmacokinetics (PK) profile of 1layer-1, 1layer-3, 2layer-1, and 2layer-3 microneedles of embodiments of the present application.

### MODE FOR INVENTION

Hereinafter, the present disclosure will be described in more detail.

The present disclosure provides a composition for preparing a microneedle.

The composition for preparing a microneedle of the present disclosure includes semaglutide and a biodegradable polymer material.

Semaglutide, as a glucagon-like peptide 1 (GLP-1) receptor agonist, is used as a therapeutic agent for type 2 diabetes and obesity, and is classified as a long-term agonist.

The biodegradable polymer material refers to a polymer material that can be degraded in a living body by body fluids, enzymes, or microorganisms.

The biodegradable polymer material may be at least one selected from the group consisting of hyaluronic acid (HA), polyvinylpyrrolidone (PVP), chondroitin, hydroxyethyl cellulose (HEC), and hydroxypropyl methyl cellulose (HPMC). In this case, compared to cases where other types of polymer materials are used, the composition for preparing a microneedle may be maintained in a good condition without being hardened or phase-separated.

The average molecular weight of HA may be from 70 to 200 kDa. The average molecular weight of chondroitin may be from 20 to 80 kDa. The average molecular weight of HEC may be from 300 to 720 kDa. The average molecular weight of HPMC may be from 10 to 80 kDa. In this case, compared to cases that fall outside the ranges above, a microneedle that maintains a strength or shape suitable for skin penetration may be prepared.

The weight ratio of semaglutide and the biodegradable polymer material may be 1:1.1 to 2.9, 1:1.2 to 2.8, 1:1.3 to 2.7, 1:1.4 to 2.6, 1:1.5 to 2.5, or 1:1.6 to 2.4.

The composition for preparing a microneedle may further include water as a solvent for dissolving semaglutide.

The composition for preparing a microneedle may further include an additive to increase the solubility of semaglutide. The additive may be, for example, at least one selected from the group consisting of disodium phosphate (Na₂HPO₄) and sodium hydroxide (NaOH). The Na₂HPO₄ may be included in an amount of 0.5 to 2 wt % relative to the total weight of the composition for preparing a microneedle. The NaOH may be included in an amount of 0.1 to 0.25 wt % relative to the total weight of the composition for preparing a microneedle.

The composition for preparing a microneedle may further include at least one selected from the group consisting of trehalose and dextran. The trehalose may be included in an amount of 0.1 to 5 wt %, 1 to 4 wt %, or 1.5 to 4.5 wt %, relative to the total weight of the composition for preparing a microneedle. The dextran may be included in an amount of 0.1 to 6 wt %, 0.5 to 5 wt %, or 1 to 4.5 wt % relative to the total weight of the composition for preparing a microneedle.

The composition for preparing a microneedle of the present disclosure can be used to prepare a microneedle maintaining a strength suitable for skin penetration while having a high loading amount of semaglutide.

The present disclosure provides a microneedle.

The microneedle of the present disclosure includes a drug-loaded layer including semaglutide and a biodegradable polymer material.

The microneedle of the present disclosure may be a soluble microneedle that releases drug as being degraded in a living body.

The microneedle of the present disclosure may be a microneedle in which semaglutide is evenly distributed throughout the structure. In this case, the microneedle may consist of the drug-loaded layer only.

Semaglutide is the same as described above.

The biodegradable polymer material may be the biodegradable polymer material included in the composition for preparing a microneedle. For example, a material having the same type or same average molecular weight as the biodegradable polymer material included in the composition for manufacturing a microneedle may be used.

The weight ratio of semaglutide and the biodegradable polymer material may be 1:1.1 to 2.9, 1:1.2 to 2.8, 1:1.3 to 2.7, 1:1.4 to 2.6, 1:1.5 to 2.5, or 1:1.6 to 2.4.

The drug-loaded layer may further include at least one selected from the group consisting of Na₂HPO₄ and NaOH. In addition, the drug-loaded layer may further include at least one selected from the group consisting of trehalose and dextran which are the aforementioned additives.

The drug-loaded layer may be formed using the composition for preparing a microneedle.

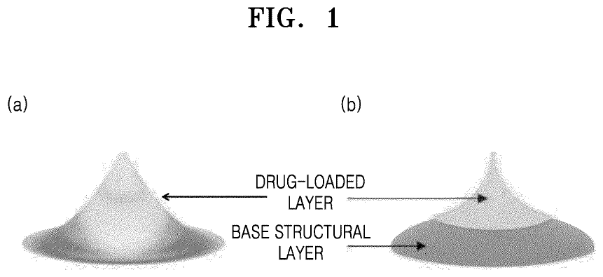

The microneedle of the present disclosure may further include a base structural layer disposed on a lower part of the drug-loaded layer and including a biodegradable polymer material. In this case, as shown in FIG. 1B, the microneedle of the present disclosure may be in a form that includes the base structural layer at the lower portion and the drug-loaded layer that is formed on the base structural layer.

The base structural layer may not include a functional material such as semaglutide.

The base structural layer may be formed using a viscous composition (second viscous composition) including a biodegradable polymer material and water.

The biodegradable polymer material of the viscous composition (second viscous composition) may be included in an amount of greater than 5 wt % and less than 20 wt %, from 6 to 19 wt %, from 7 to 18 wt %, or from 8 to 15 wt %, relative to the total weight of the viscous composition. In this case, compared to cases that fall outside the ranges above, the base structural layer may be formed with a width and height capable of forming the drug-loaded layer of an appropriate volume at the upper portion of the base structural layer.

The biodegradable polymer material of the base structural layer may be at least one selected from the group consisting of HA and carboxymethyl cellulose. In this case, the adhesion between the base structural layer and the drug-loaded layer including semaglutide is superior to that in cases where different types of polymer materials are used.

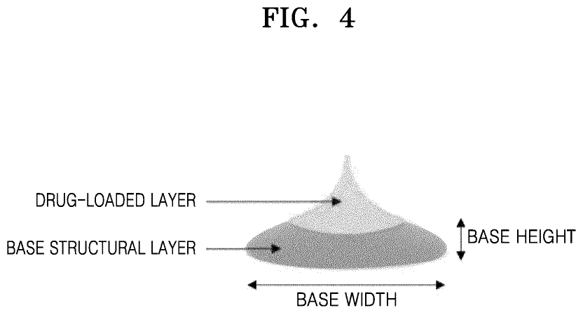

The maximum width of the base structural layer may be from 800 μm to 1,400 μm. The maximum width refers to the diameter of the bottom surface of the formed base structural layer (see FIG. 4).

When the maximum width of the base structural layer is less than 800 μm, the area of the bottom surface of the base structural layer is small such that the adhesion between the support on which the microneedle is formed and the base structural layer may be reduced, or the area of the top surface of the base structural layer is small such that the loading amount of semaglutide in the drug-loaded layer formed on the base structural layer may be reduced. When the maximum width of the base structural layer is greater than 1,400 μm, the area of the bottom surface of the base structural layer is large such that the microneedle may have a difficulty in penetrating the skin.

The height of the base structural layer may be from 150 μm to 250 μm (see FIG. 4).

When the height of the base structural layer is less than 150 μm, semaglutide loaded in the lower portion of the drug-loaded layer may not be delivered into the body, but may remain, causing a loss. When the height of the base structural layer is greater than 250 μm, it is difficult to form the drug-loaded layer on the base structural layer, and the loading amount of semaglutide loaded in the drug-loaded layer may decrease.

In an embodiment, the length of the microneedle may be from 700 to 800 μm from the support on which the microneedle is formed. In this case, a sufficient amount of semaglutide may be loaded into the microneedle, and when applied to the skin, the microneedle does not affect nerve cells and thus does not cause pain.

In an embodiment, the tip diameter of the microneedle may be from 50 to 90 μm. In this case, a sufficient amount of semaglutide may be loaded into the microneedle, and the microneedle may still effectively penetrate the skin.

In an embodiment, the strength of the microneedle may be 0.058 N or more. In this case, the needle may effectively penetrate the skin without breaking or bending when applied to the skin.

The microneedle of the present disclosure may maintain a strength suitable for penetrating the skin while having high loading of semaglutide, and thus a sufficient amount of drug may be delivered into the body even with a small amount. The microneedle of the present disclosure may reduce the risk of bleeding, risk of infection, pain, fear, etc. compared to a self-injectable therapeutic agent, thereby increasing convenience of administration for patients and reducing medical waste due to biodegradability in the body.

When the microneedle of the present disclosure includes the drug-loaded layer and the base structural layer disposed on the lower part of the drug-loaded layer, the residual amount of semaglutide may be small, thereby reducing the loss of semaglutide.

The present disclosure provides a method of preparing a microneedle.

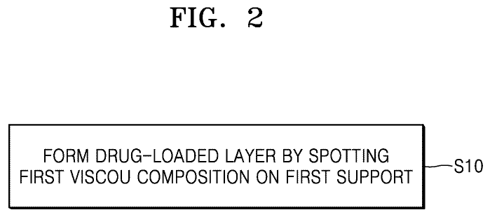

The method of preparing a microneedle of the present disclosure includes: forming a drug-loaded layer by spotting a first viscous composition including semaglutide and a biodegradable polymer material on a first support. (Process S10 of FIG. 2)

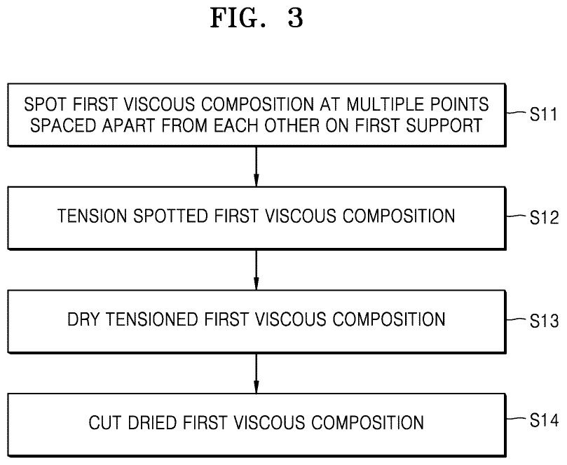

The forming of the drug-loaded layer may include: spotting the first viscous composition at multiple points spaced apart from each other on the first support; tensioning the spotted first viscous composition; and drying the tensioned first viscous composition. In addition, in this case, the method may further include cutting the dried first viscous composition. (Processes S11 to S14 of FIG. 3)

The tensioning of the first viscous composition may be performed by contacting a second support with the spotted first viscous composition and subsequently spacing the first support and the second support apart.

The first support may be, for example, selected from the group consisting of HA or a pharmaceutically acceptable salt thereof, carboxymethyl cellulose, PVP, polyvinyl alcohol, polylactic glycolic acid, gelatin, collagen, chitosan, and a mixture thereof.

The second support is the same as the aforementioned first support.

The cutting of the first viscous composition may be performed by spacing the first support and the second support farther apart, or by using an appropriate device by a person skilled in the art.

The first viscous composition is the same as the composition for preparing a microneedle of the present disclosure or the viscous composition used to form the drug-loaded layer, and thus, its description will be omitted to avoid duplication.

The spotting refers to discharging the first viscous composition in the form of droplets. The spotting may be performed with a dispenser, specifically with an air-jet dispenser.

By the processes above, a microneedle containing semaglutide evenly distributed throughout the structure may be prepared. The microneedle prepared by the processes above may maintain a strength suitable for penetrating the skin while having high loading of semaglutide, and thus a sufficient amount of drug may be delivered into the body even with a small amount.

In addition, the method of preparing a microneedle of the present disclosure may further include forming a base structural layer by spotting a second viscous composition including a biodegradable polymer material on the first support, before the spotting of the first viscous composition on the first support. In this case, the first viscous composition may be spotted on the base structural layer.

When the forming of the base structural layer is further included, a microneedle, in which a base structural layer is included at the lower portion by the process above and a drug-loaded layer is formed on the base structural layer, may be prepared. The microneedle prepared by the process above has a small residual amount of semaglutide, thereby reducing loss of semaglutide.

The forming of the base structural layer may include: spotting the second viscous composition at multiple points spaced apart from each other on the first support; and drying the spotted second viscous composition.

The spotting of the second viscous composition may be performed in a way that the base structural layer formed using the second viscous composition has preferred maximum width and height. The preferred maximum width and height are the same as described in connection with the base structural layer of the microneedle of the present disclosure.

The drying of the second viscous composition may be performed under conditions that the upper portion of the base structural layer formed using the second viscous composition does not collapse or break. For example, the drying may be performed by blowing air or under conditions maintained at a low humidity (15% or less).

The second viscous composition may not include a functional material such as semaglutide.

The second viscous composition is the same as the viscous composition used to form the base structural layer of the microneedle of the present disclosure, and thus its description will be omitted to avoid duplication.

As described above, the microneedle prepared by the preparation method of the present disclosure may maintain a strength suitable for penetrating the skin while having high loading of semaglutide, and thus a sufficient amount of drug may be delivered into the body even with a small amount. In addition, the microneedle prepared by the preparation method of the present disclosure and including the drug-loaded layer and the base structural layer has a small residual amount of semaglutide, thereby reducing loss of semaglutide.

Hereinafter, the present disclosure will be described in detail with reference to embodiments.

### EXAMPLES

### 1. Preparation and Evaluation of Composition for Preparing Microneedle

### (1) Preparation of Composition for Preparing Microneedle

Components listed in Table 1 below were mixed in specified amounts (wt %) together with 1 wt % of disodium phosphate (Na₂HPO₄) and 0.2 wt % of sodium hydroxide (NaOH) to prepare a composition for forming a microneedle. The Na₂HPO₄ and NaOH were added to increase the solubility of semaglutide. The prepared composition for preparing a microneedle is also referred to as a composition for preparing a drug-loaded layer described below.
(HA: hyaluronic acid, PVP: polyvinylpyrrolidone, C: chondroitin, HEC: hydroxyethyl cellulose, HPMC: hydroxypropyl methyl cellulose)

TABLE 1

| | Biodegradable polymer | | | | |
|---|---|---|---|---|---|
| Compo- | | Amount | Semaglutide | Water | Additive |
| sition | Type | (wt %) | (wt %) | (wt %) | (wt %) |
| A-1 | HA | 16 | 10 | 72.8 | — |
| A-2 | HA, PVP | 19 | 10 | 66.8 | 3 (trehalose) |
| A-3 | C, HEC | 24 | 10 | 64.8 | — |
| A-4 | HPMC | 23 | 10 | 65.8 | — |
| A-5 | HA | 16 | 10 | 68.8 | 4 (Dextran) |
| A-6 | HA | 16 | 10 | 70.8 | 2 (Dextran) |
| A-7 | HA | 10 | 20 | 68.8 | — |
| A-8 | HA | 16 | 20 | 62.8 | — |
| A-9 | HA | 16 | 25 | 57.8 | — |
| A-10 | HA, PVP | 16.5 | 20 | 60.3 | 2 (trehalose) |
| A-11 | HA, PVP | 15.5 | 20 | 61.3 | 2 (trehalose) |
| A-12 | HA, PVP | 12 | 20 | 64.8 | 2 (trehalose) |
| A-13 | HA, PVP | 21 | 20 | 57.8 | — |
| A-14 | HA | 31.2 | 10 | 50.6 | 7 (Dextran) |
| A-15 | HA | 30 | 10 | 50.8 | 8 (Dextran) |
| A-16 | HA | 10 | 20 | 62.8 | 6 (Dextran) |

### (2) Evaluation of Physical Properties of Composition for Preparing Microneedle

After the composition for preparing a microneedle prepared in Section 1-(1) was left at room temperature for one hour, the state of the composition was observed. When the composition was uniformly mixed and present in a liquid state, it is described as “good,” when the composition is hardened at room temperature, it is described as “hardened,” and when the composition is present in a phase-separated state, it is described as “phase-separated”.

When the composition for preparing a microneedle prepared in Section 1 is present in a liquid state at room temperature, the viscosity (cP) of the composition at 25° C. was measured using a rotational automatic viscometer manufactured by Brookfield.

Afterwards, using the composition for forming a microneedle present in a liquid state, a microneedle was prepared according to a method described in Section 3, and it was observed whether a microneedle having a tip shape was formed. When a microneedle was formed, it is marked with an “O,” and when a microneedle was not formed, it is marked with an “X”.

The results of evaluating physical properties of the composition are shown in Table 2.

TABLE 2

| | Composition | Composition | Formation of |
|---|---|---|---|
| Composition | state | viscosity (cP) | microneedle |
| A-1 | Good | 45,000 | ◯ |
| A-2 | Good | 44,000 | ◯ |
| A-3 | Good | 43,110 | ◯ |
| A-4 | Good | 40,000 | ◯ |
| A-5 | Good | 48,310 | ◯ |
| A-6 | Good | 46,400 | ◯ |
| A-7 | Good | 20,000 | X |
| A-8 | Hardened | — | — |
| A-9 | Hardened | — | — |
| A-10 | Phase-separated | — | — |
| A-11 | Hardened | — | — |
| A-12 | Hardened | — | — |
| A-13 | Good | 28,000 | X |
| A-14 | Hardened | — | — |
| A-15 | Hardened | — | — |
| A-16 | Good | 24,580 | X |

### 2. Preparation and Evaluation of Microneedles

### (1) 1 Layer Microneedles

### 1) Preparation of 1 Layer Microneedles

A composition for preparing a microneedle listed in Table 3 below was spotted on a support and tensioned, and then the tensioned composition was dried by blowing air. The composition for preparing a microneedle solidified by drying was cut to form microneedles. The number of microneedles formed on the support is as shown in Table 3 below. The microneedles prepared by the method described herein are those in which a drug is evenly distributed throughout the needle structure, and are in the form including only a drug-loaded layer. For convenience, needles of this type are denoted as 1 layer.

TABLE 3

| | Composition for | |
|---|---|---|
| Microneedle | preparing microneedle | Count |
| 1layer-1 | A-1 | 25 |
| 1layer-2 | A-1 | 100 |
| 1layer-3 | A-2 | 25 |
| 1layer-4 | A-2 | 100 |
| 1layer-5 | A-3 | 25 |
| 1layer-6 | A-3 | 100 |
| 1layer-7 | A-4 | 25 |
| 1layer-8 | A-4 | 100 |
| 1layer-9 | A-5 | 25 |
| 1layer-10 | A-5 | 100 |
| 1layer-11 | A-6 | 25 |
| 1layer-12 | A-6 | 100 |

### 2) Evaluation of 1 Layer Microneedles

The microneedles prepared in Section 2-(1)-1) were observed using an optical microscope to measure the length and tip diameter of the microneedles. The measurement results are shown in Table 4 below.

The compressive strength of three microneedles in the center among the microneedles was measured using a tensile and compression testing device. The strength was measured repeatedly for six samples prepared using the same composition, and the average strength value was then calculated. The strength of the microneedles is as shown in Table 4 below.

TABLE 4

| | Needle | Tip | Needle |
|---|---|---|---|
| Microneedle | length (μm) | diameter (μm) | strength (N) |
| 1layer-1 | 776 | 82 | 0.192 |
| 1layer-2 | 770 | 79 | 0.184 |
| 1layer-3 | 710 | 80 | 0.615 |
| 1layer-4 | 701 | 73 | 0.522 |
| 1layer-5 | 721 | 88 | 0.81 |
| 1layer-6 | 711 | 90.2 | 0.72 |
| 1layer-7 | 718 | 78 | 0.569 |
| 1layer-8 | 707 | 79.6 | 0.583 |
| 1layer-9 | 799 | 77 | 0.430 |
| 1layer-10 | 728 | 79 | 0.102 |
| 1layer-11 | 733 | 72 | 0.299 |
| 1layer-12 | 736 | 71 | 0.702 |

### (2) 2 Layer Microneedles

### 1) Preparation of 2 Layer Microneedles

To prepare a microneedle (2 layer microneedle) further including a base structural layer disposed on the lower part of a drug-loaded layer, components listed in Table 5 below were mixed in specified amounts (wt %) to prepare a composition for forming a base structural layer.
(HA: hyaluronic acid, CMC: carboxymethyl cellulose)

TABLE 5

| | Biodegradable polymer | | |
|---|---|---|---|
| | | Amount | Water amount |
| Composition | Type | (wt %) | (wt %) |
| B-1 | HA, CMC | 8 | 92 |
| B-2 | HA, CMC | 10 | 90 |
| B-3 | HA, CMC | 15 | 85 |
| B-4 | HA, CMC | 20 | 80 |
| B-5 | HA, CMC | 5 | 95 |

Subsequently, 2-layer microneedles were prepared using the composition for preparing a microneedle prepared in Section 1-(1) (Table 1) and the composition for preparing a base structural layer of Table 5.

The composition for preparing a base structural layer described in Table 6 below was spotted at multiple points on a support, and then dried by blowing air at room temperature, so as to form a plurality of base structural layers (each having a width of 1000 μm and a height of 200 μm). The number of base structural layers formed on the support is as described in Table 6 below.

The composition for preparing a microneedle listed in Table 6 (for forming a drug-loaded layer) was spotted on each of the plurality of base structural layers and tensioned, and then the tensioned composition was dried by blowing air. The composition for preparing a microneedle solidified by drying was cut to form a drug-loaded layer, thereby preparing 2 layer microneedles.

TABLE 6

| Microneedle | Base structural layer | Drug-loaded layer | Count |
|---|---|---|---|
| 2layer-1 | B-1 | A-1 | 25 |
| 2layer-2 | B-1 | A-1 | 100 |
| 2layer-3 | B-1 | A-2 | 25 |
| 2layer-4 | B-1 | A-2 | 100 |
| 2layer-5 | B-1 | A-3 | 25 |
| 2layer-6 | B-1 | A-3 | 100 |
| 2layer-7 | B-1 | A-4 | 25 |
| 2layer-8 | B-1 | A-4 | 100 |
| 2layer-9 | B-1 | A-5 | 25 |
| 2layer-10 | B-1 | A-5 | 100 |
| 2layer-11 | B-1 | A-6 | 25 |
| 2layer-12 | B-1 | A-6 | 100 |
| 2layer-13 | B-2 | A-1 | 25 |
| 2layer-14 | B-2 | A-1 | 100 |
| 2layer-15 | B-3 | A-5 | 25 |
| 2layer-16 | B-3 | A-5 | 100 |
| 2layer-17 | B-4 | A-1 | 25 |
| 2layer-18 | B-5 | A-1 | 25 |

### 2) Evaluation of 2 Layer Microneedles

The microneedles prepared in Section 2-(2)-1) were observed using an optical microscope to measure the length and tip diameter of the microneedles. The measurement results are shown in Table 7 below.

The compressive strength of three microneedles in the center among the microneedles was measured using a tensile and compression testing device. The strength was measured repeatedly for six samples prepared using the same composition, and then the average strength value was calculated. The strength of the microneedles is as shown in Table 7 below.

TABLE 7

| | Needle | Tip | Needle |
|---|---|---|---|
| Microneedle | length (μm) | diameter (μm) | strength (N) |
| 2layer-1 | 794 | 83.7 | 0.707 |
| 2layer-2 | 769 | 83 | 0.643 |
| 2layer-3 | 736 | 65 | 0.234 |
| 2layer-4 | 721 | 70 | 0.250 |
| 2layer-5 | 770 | 53.7 | 0.463 |
| 2layer-6 | 762 | 55.3 | 0.53 |
| 2layer-7 | 719 | 78 | 0.087 |
| 2layer-8 | 723 | 74.2 | 0.094 |
| 2layer-9 | 792 | 63 | 0.462 |
| 2layer-10 | 732 | 70 | 0.146 |
| 2layer-11 | 800 | 68 | 0.321 |
| 2layer-12 | 775 | 66 | 0.684 |
| 2layer-13 | 782 | 77 | 0.490 |
| 2layer-14 | 751 | 65 | 0.404 |
| 2layer-15 | 793 | 78 | 0.098 |
| 2layer-16 | 799 | 62 | 0.323 |
| 2layer-17 | 863 | 95 | 0.842 |
| 2layer-18 | 584 | 54 | 0.061 |

### 3. Evaluation of Semaglutide PK Profile of Microneedle and Area Under Curve Per Dose of Microneedle

The area under curve (AUC) per dose of 1 layer-1 and 1 layer-3 microneedles of Table 3 above and 2 layer-1 and 2 layer-3 microneedles of Table 6 above was evaluated.

Specifically, each of the aforementioned microneedles was inserted into shaved minipigs aged 8 to 13 months, and blood samples were collected 0, 3, 6, 9, 18, 24, 30, 48, 72, 96, 120, 144, and 168 hours later. The blood samples were centrifuged to obtain serum, which was then analyzed by LC/MS-MS (Model: Triple Quad™M 5500, Manufacturer: Agilent technology (LC) AB SCIEX (MS)) to detect the concentration of semaglutide in the blood.

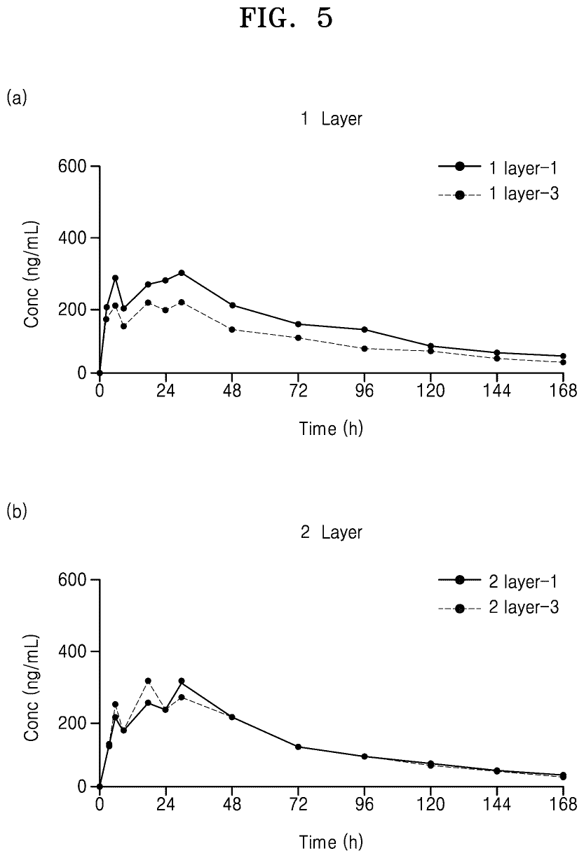

The blood concentration of semaglutide according to the blood sampling time was plotted on a coordinate plate with time on the x-axis and blood concentration on the y-axis to obtain a pharmacokinetics (PK) profile. The AUC of semaglutide was calculated by integrating the obtained PK profile, and the administered dose of semaglutide to the minipigs was adjusted to evaluate the microneedles (FIG. 5).

The AUC indicates the degree of drug bioavailability, and reflects the total amount of active drug reaching the systemic circulation. As shown in Table 8 below, all the 1 layer-1, 1 layer-3, 2 layer-1, and 2 layer-3 microneedles showed an AUC of 10,000 ng·h/mL or more, confirming that all needles had excellent bioavailability. Due to the base structural layer formed at the lower portion of the 2 layer microneedle, the 2 layer needle exhibited a relatively larger AUC per dose than the 1 layer needle.

TABLE 8

| | Dose | Area under | Area under |
|---|---|---|---|
| Microneedle | (mg) | curve (ng · h/mL) | curve per dose |
| 2layer-1 | 1.36 | 20042 | 14737 |
| 2layer-3 | 1.52 | 20826 | 13701 |
| 1layer-1 | 3.12 | 26617 | 8531 |
| 1layer-3 | 2.78 | 17567 | 6319 |

### 4. Evaluation of Residual Amount of Semaglutide in Microneedle

1 layer-2, 1 layer-4, and 1 layer-6 microneedles of Table 3 above and 2 layer-2, 2 layer-4, and 2 layer-6 microneedles of Table 6 above were attached to pig skin and removed after 8 hours, and then the residual amount (%) of semaglutide remaining on a patch was evaluated.

Specifically, the microneedles were inserted into shaved minipigs aged less than 12 months and removed after 8 hours. A patch attachment site was wiped with gauze which was then analyzed together with the removed patch using HPLC (manufacturer: Agilent) to calculate the concentration of semaglutide (Table 9).

As shown in Table 9, due to the base structural layer formed at the lower portion of the 2 layer microneedle, the 2 layer-type needle had a relatively small residual amount than the 1 layer-type needle.

TABLE 9

| Microneedle | Residual amount (%) |
|---|---|
| 2layer-2 | 25.27 |
| 2layer-4 | 25.20 |
| 2layer-6 | 40.68 |
| 1layer-2 | 54.29 |
| 1layer-4 | 52.29 |
| 1layer-6 | 69.92 |

1. A composition for preparing a microneedle, comprising semaglutide and a biodegradable polymer material.

2. The composition for preparing a microneedle of claim 1, wherein the biodegradable polymer material is at least one selected from the group consisting of hyaluronic acid, polyvinylpyrrolidone, chondroitin, hydroxyethyl cellulose, and hydroxypropyl methylcellulose.

3. The composition for preparing a microneedle of claim 1, wherein a weight ratio of the semaglutide and the biodegradable polymer material is 1:1.1 to 2.9.

4. The composition for preparing a microneedle of claim 1, further comprising at least one selected from the group consisting of disodium phosphate (Na₂HPO₄) and sodium hydroxide (NaOH).

5. The composition for preparing a microneedle of claim 1, further comprising at least one selected from the group consisting of trehalose and dextran.

6. A microneedle comprising a drug-loaded layer including semaglutide and a biodegradable polymer material.

7. The microneedle of claim 6, wherein the biodegradable polymer material of the drug-loaded layer is at least one selected from the group consisting of hyaluronic acid, polyvinylpyrrolidone, chondroitin, hydroxyethyl cellulose, and hydroxypropyl methylcellulose.

8. The microneedle of claim 6, wherein a weight ratio of the semaglutide and the biodegradable polymer material in the drug-loaded layer is 1:1.1 to 2.9.

9. The microneedle of claim 6, further comprising a base structural layer that is disposed on a lower part of the drug-loaded layer and includes a biodegradable polymer material.

10. The microneedle of claim 9, wherein the base structural layer is formed using a viscous composition including a biodegradable polymer material and water.

11. The microneedle of claim 10, wherein the biodegradable polymer material of the viscous composition is included in an amount of 8 to 15 wt % based on a total weight of the viscous composition.

12. The microneedle of claim 9, wherein the biodegradable polymer material of the base structural layer is at least one selected from the group consisting of hyaluronic acid and carboxymethyl cellulose.

13. A method of preparing a microneedle, comprising: forming a drug-loaded layer by spotting a first viscous composition including semaglutide and a biodegradable polymer material on a first support.

14. The method of preparing a microneedle of claim 13, wherein the forming of the drug-loaded layer comprises: spotting a first viscous composition at multiple points spaced apart from each other on the first support; tensioning spotted first viscous composition; and drying tensioned first viscous composition.

15. The method of preparing a microneedle of claim 14, wherein the tensioning of the first viscous composition is performed by contacting a second support with spotted first viscous composition and subsequently spacing the first support and the second support apart.

16. The method of preparing a microneedle of claim 14, wherein the forming of the drug-loaded layer further comprises cutting dried first viscous composition.

17. The method of preparing a microneedle of claim 13, wherein the biodegradable polymer material of the first viscous composition is at least one selected from the group consisting of hyaluronic acid, polyvinylpyrrolidone, chondroitin, hydroxyethyl cellulose, and hydroxypropyl methylcellulose.

18. The method of preparing a microneedle of claim 13, wherein a weight ratio of the semaglutide and the biodegradable polymer material in the first viscous composition is 1:1.1 to 2.9.

19. The method of preparing a microneedle of claim 13, further comprising: forming a base structural layer by spotting a second viscous composition including a biodegradable polymer material on the first support, before the spotting of the first viscous composition on the first support, wherein the first viscous composition is spotted on the base structural layer.

20. The method of preparing a microneedle of claim 19, wherein the forming of the base structural layer comprises: spotting the second viscous composition at multiple points spaced apart from each other on the first support; and drying spotted second viscous composition.

21. The method of preparing a microneedle of claim 19, wherein the second viscous composition includes a biodegradable polymer material and water.

22. The method of preparing a microneedle of claim 21, wherein the biodegradable polymer material of the second viscous composition is included in an amount of 8 to 15 wt % based on a total weight of the second viscous composition.

23. The method of preparing a microneedle of claim 19, wherein the biodegradable polymer material of the second viscous composition is at least one selected from the group consisting of hyaluronic acid and carboxymethyl cellulose.
